(19) United States
(12) Patent Application Publication
(10) Pub. No.: US 2019/0247591 A1
(43) Pub. Date: Aug. 15, 2019

(54) SINGLE USE SELF-DISABLING SYRINGE

(71) Applicant: Chung-Sik KIM, Incheon (KR)

(72) Inventor: Chung-Sik KIM, Incheon (KR)

(21) Appl. No.: 16/136,264

(22) Filed: Sep. 20, 2018

(30) Foreign Application Priority Data
Feb. 13, 2018 (KR) 20-2018-0000696

(51) Int. Cl. A61M 5/50

(52) U.S. Cl. CPC A61M 5/5066; A61M 2005/5073

(57) ABSTRACT

Disclosed is a single use self-disabling syringe capable of preventing the syringe from being reused after the syringe is used once on a patient, thereby preventing syringe reuse. The single use self-disabling syringe includes: a barrel having a needle attached to a tip end thereof and a fluid containing space defined therein, the fluid containing space being open at a rear end of the barrel; a plunger contained in the barrel and configured to draw in and expel a fluid; and a syringe reuse prevention means provided in the barrel along an inner surface thereof in an axial direction and configured to tear a part of the plunger so as to prevent the syringe from being reused.

### BACKGROUND OF THE INVENTION

### Field of the Invention

The present invention relates generally to a single use self-disabling syringe and, more particularly, to a single use self-disabling syringe capable of preventing the syringe from being reused after the syringe is used once on a patient, thereby preventing syringe reuse.

### Description of the Related Art

In general, among everyday items, there are many products that are prohibited by law from being reused after being used once for the purpose of hygiene or maintenance of social safety. However, there are frequent cases of reusing a product that is prohibited from being reused, mainly for economic reasons.

Accordingly, it cannot be said that it is a responsible action not to provide practical measures but only to enact a law to prohibit reuse. In addition to enacting the law, it may be necessary to make the product prohibited from being reused be self-disable itself by using a technical device such that a single use product cannot be reused even when a user attempts to reuse the product.

In particular, disposable syringes, which must be discarded after one use in order to prevent infection, are designed such that a configuration of a syringe itself is reusable as much as possible. In some cases, disposable syringes may be sterilized in hot water for reuse, and even syringes that have not undergone sterilization may be reused, leading to serious side effects.

The reuse of disposable syringes is common in the world's poorest countries, and there are cases of unscrupulous behavior by medical practitioners who reuse disposable syringes more than once. The reuse of syringes has caused spread of various infectious, causing serious social problems.

In order to solve such a problem, recently, many types of disposable syringes have been developed for reuse prevention. However, the disposable syringes for reuse prevention in the related art are problematic in that a technical configuration thereof is complicated and manufacturing cost is high, resulting in poor economical efficiency.

For example, in some products, since the technical configuration for reuse prevention is complicated and thus a product price is higher than that of a typical disposable syringe for which a reuse prevention configuration is not provided, the products are not used in a medical field.

The foregoing is intended merely to aid in the understanding of the background of the present invention, and is not intended to mean that the present invention falls within the purview of the related art that is already known to those skilled in the art.

### DOCUMENTS OF RELATED ART

(Patent document 1) Korean Patent No. 10-1660906 (Sep. 22, 2016)

### SUMMARY OF THE INVENTION

Accordingly, the present invention has been made keeping in mind the above problems occurring in the related art, an objective of the present invention is to provide a single use self-disabling syringe capable of preventing the syringe from being reused after the syringe is used once on a patient, thereby preventing syringe reuse.

Another objective of the present invention is to provide a single use self-disabling syringe configured such that a plunger that performs a piston motion in a barrel is torn upon fluid expulsion, thereby preventing syringe reuse.

Yet another objective of the present invention is to provide a single use self-disabling syringe that is simple in construction and easy to manufacture.

Still another objective of the present invention is to provide a single use self-disabling syringe, wherein a disposable syringe is easily used but is prevented from being reused after being used once, thereby solving various problems caused due to syringe reuse.

In order to achieve the above objectives, according to one aspect of the present invention, there is provided a single use self-disabling syringe, including: a barrel having a needle attached to a tip end thereof and a fluid containing space defined therein, the fluid containing space being open at a rear end of the barrel; a plunger contained in the barrel and configured to draw in and expel a fluid; and a syringe reuse prevention means provided in the barrel along an inner surface thereof in an axial direction and configured to tear a part of the plunger so as to prevent the syringe from being reused.

Furthermore, the syringe reuse prevention means may be provided as a protrusion protruding to be inclined from a first end to a second end thereof.

Furthermore, the syringe reuse prevention means may be provided as a wedge-shaped protrusion inclined from a first end to a second end thereof such that the first end has a height higher than a height of the second end, and the protrusion may have a wedge shape in cross section, inclined downward from a middle top to left and right ends of the cross section.

Furthermore, the protrusion may have a “\hat{ }”-shaped wedge shape in cross section.

Furthermore, the protrusion may protrude linearly from the inner surface of the barrel in the axial direction at at least two positions.

The present invention can provide the single use self-disabling syringe capable of preventing the syringe from being reused after the syringe is used once on a patient, thereby preventing syringe reuse.

Furthermore, the present invention can provide the single use self-disabling syringe configured such that the plunger that performs a piston motion in the barrel is torn upon fluid expulsion, thereby preventing syringe reuse.

Furthermore, the present invention can provide the single use self-disabling syringe that is simple in construction and easy to manufacture.

Furthermore, the present invention can provide the single use self-disabling syringe, wherein a disposable syringe is easily used but is prevented from being reused after being used once, thereby solving various problems caused due to syringe reuse.

### BRIEF DESCRIPTION OF THE DRAWINGS

The above and other objects, features and other advantages of the present invention will be more clearly understood from the following detailed description when taken in conjunction with the accompanying drawings, in which:

FIGS. 1 to 5 show an embodiment of a single use self-disabling syringe according to the present invention, wherein

FIG. 1 is an overall partial sectional exemplary view showing the single use self-disabling syringe according to the present invention;

FIG. 2 is a front view showing a barrel of the single use self-disabling syringe according to the present invention;

FIG. 3 is a left/right side view showing the barrel of the single use self-disabling syringe according to the present invention;

FIG. 4 is a sectional exemplary view showing the barrel of the single use self-disabling syringe according to the present invention; and

FIG. 5 is a view showing a use state before and after use of the single use self-disabling syringe according to the present invention.

### DETAILED DESCRIPTION OF THE INVENTION

Reference will now be made in greater detail to exemplary embodiments of the present invention, examples of which are illustrated in the accompanying drawings. The exemplary embodiments described hereinbelow are provided for fully conveying the scope and spirit of the invention to those skilled in the art, so it should be understood that the embodiments may be changed to a variety of embodiments and the scope and spirit of the invention are not limited to the embodiments described hereinbelow.

Hereinbelow, exemplary embodiments of the present invention will be described in detail with reference to the accompanying drawings. Like reference numerals are used to identify like elements throughout different drawings. Further, in the following description, if it is decided that the detailed description of known function or configuration related to the invention makes the subject matter of the invention unclear, the detailed description is omitted.

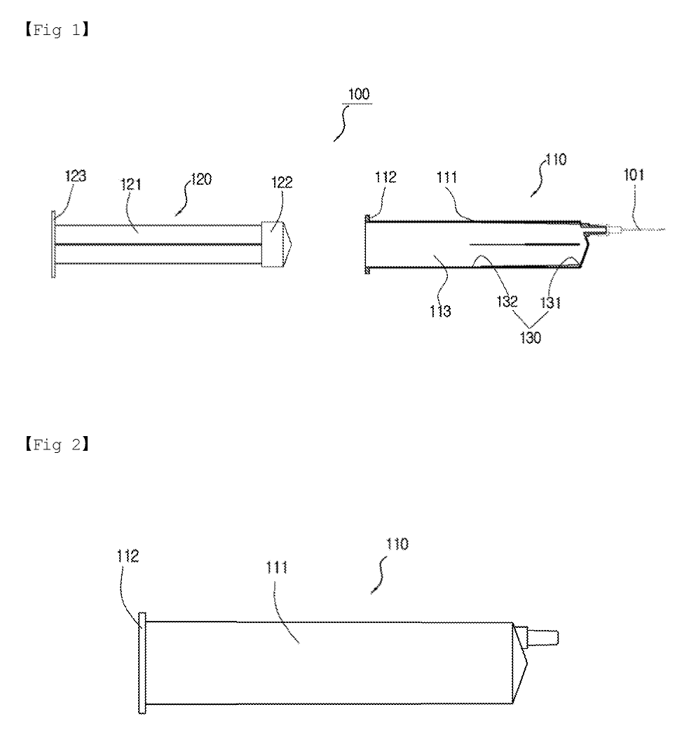

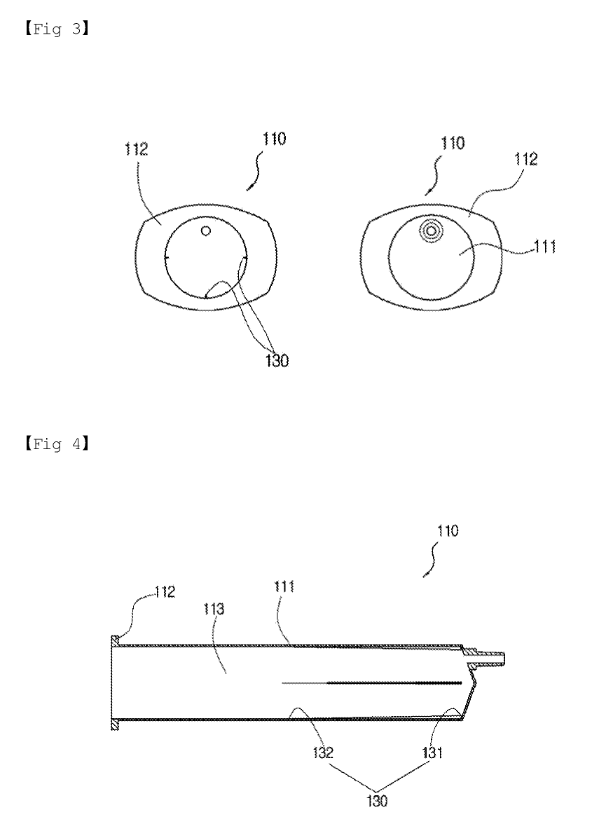

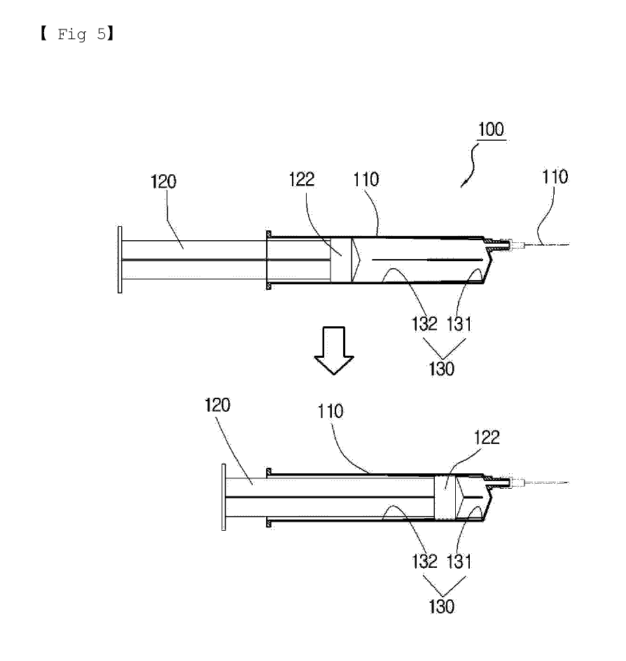

FIGS. 1 to 5 are views showing an embodiment of a single use self-disabling syringe according to the present invention.

Referring to FIGS. 1 to 4, a single use self-disabling syringe 100 according to the present invention includes a barrel 110 having a needle 101 attached to a tip end thereof and a fluid containing space defined inside the barrel, the fluid containing space being open at a rear end of the barrel; a plunger 120 contained in the barrel 110 and configured to draw in and expel a fluid; and a syringe reuse prevention means provided in the barrel in an axial direction and configured to tear a part of the plunger so as to prevent the syringe from being reused.

The barrel 110 may further include a barrel body 111 having a substantially cylindrical shape and to which the needle 101 is attached while creating a seal around a tip end of the barrel body; a finger flange 112 radially protruding from a rear end of the barrel body 111 at a predetermined height and operable by a user's hand; and the syringe reuse prevention means protruding along an inner surface 113 of the barrel body 111 and configured to tear the part of the plunger 120.

The plunger 120 is contained in the barrel 110 so as to draw in and expel the fluid and includes a plunger body 121 provided in the axial direction;

an elastic rubber 122 provided at a tip end of the plunger body 121 and configured to advance and retract along the inner surface of the barrel body so as to elastically maintain a sealing force; and a thumb seat 123 radially protruding from a rear end of the plunger body 121 and operable by a user's hand.

Meanwhile, the syringe reuse prevention means may be provided as a protrusion 130 protruding to be inclined from a first end 131 to a second end 132.

The syringe reuse prevention means may be provided as a wedge-shaped protrusion 130 inclined from the first end 131 to the second end 132 such that the first end has a height higher than a height of the second end.

Herein, it is preferable that the protrusion 130 has a wedge shape in cross section, inclined downward from a middle top to left and right ends of the cross section.

It is preferable that the protrusion 130 has a “\hat{ }”-shaped wedge shape in cross section.

It is preferable that the syringe reuse prevention means protrudes linearly from the inner surface of the barrel 110 in the axial direction at at least two positions. In the drawings, three protrusions 130 are shown.

Hereinafter, an operation effect according to the single use self-disabling syringe according to the present invention will be described, and a configuration and reference numerals will be described by applying the components described previously.

Referring to FIG. 6, in the use of the single use self-disabling syringe 100, the plunger 120 is pulled backward to draw in a desired amount of the fluid into the space defined in the barrel 110 and stands by.

Next, in the use of the syringe on a patient, the plunger 120 is pushed forward against the tip end of the barrel 110 to expel the fluid from the syringe whereby the use of the syringe is completed.

Herein, the elastic rubber 122 of the plunger 120 is advanced along a top edge of the wedge-shaped protrusion 130 protruding from the inner surface 113 of the barrel as the syringe reuse prevention means, whereby the part of the elastic rubber 122 is torn.

In other words, the elastic rubber 122 of the plunger 120 is advanced along a middle top edge of the wedge-shaped protrusion 130, whereby the surface of the elastic rubber 122 is cut.

In other words, the elastic rubber 122 of the plunger 120 is advanced along the protrusion 130 to be torn while at the same time the fluid is expelled from the syringe by the plunger 120. This makes it possible to check whether the elastic rubber 122 of the plunger 120 is destroyed or not and to prevent the syringe from being reused even when a user attempts to reuse the same.

As described above, the single use self-disabling syringe according to the present invention can prevent the syringe from being reused after the syringe is used once on a patient, thereby preventing syringe reuse. This is achieved by tearing the plunger that performs a piston motion in the barrel, thereby providing the single use self-disabling syringe for reuse prevention.

Although embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. A single use self-disabling syringe, comprising:
a barrel having a needle attached to a tip end thereof and a fluid containing space defined therein, the fluid containing space being open at a rear end of the barrel;
a plunger contained in the barrel and configured to draw in and expel a fluid; and
a syringe reuse prevention means provided in the barrel along an inner surface thereof in an axial direction and configured to tear a part of the plunger so as to prevent the syringe from being reused.

2. The single use self-disabling syringe of claim 1, wherein the syringe reuse prevention means is provided as a protrusion protruding to be inclined from a first end to a second end thereof.

3. The single use self-disabling syringe of claim 1, wherein the syringe reuse prevention means is provided as a wedge-shaped protrusion inclined from a first end to a second end thereof such that the first end has a height higher than a height of the second end, and
the protrusion has a wedge shape in cross section, inclined downward from a middle top to left and right ends of the cross section.

4. The single use self-disabling syringe of claim 3, wherein the protrusion has a “\hat{ }”-shaped wedge shape in cross section.

5. The single use self-disabling syringe of claim 1, wherein the protrusion protrudes linearly from the inner surface of the barrel in the axial direction at at least two positions.
